(19) United States
(12) Patent Application Publication
(10) Pub. No.: US 2022/0257817 A1
(43) Pub. Date: Aug. 18, 2022

(54) DOOR HANDLE WITH DISINFECTANT FUNCTION

(71) Applicant: Dogus KOYUNCU, Ankara (TR)

(72) Inventor: Dogus KOYUNCU, Ankara (TR)

(21) Appl. No.: 17/623,620

(22) PCT Filed: Aug. 21, 2020
(86) PCT No.: PCT/TR2020/050738

§ 371 Date: Dec. 29, 2021

(30) Foreign Application Priority Data
Aug. 23, 2019 (TR) 2019/12776
Aug. 23, 2019 (TR) 2019/12779
Mar. 6, 2020 (TR) 2020/03536

(51) Int. Cl. A61L 2/18; E05B 1/00

(52) U.S. Cl. CPC A61L 2/18; A61L 2202/15; A61L 2202/14; E05B 1/0069

(57) ABSTRACT

A door handle with a disinfectant function includes a liquid container containing a disinfectant liquid. The liquid container is embedded into an inner body of a door. The door handle with the disinfectant function enables to clean door handles used in public and private places such as hospitals, health care centers, public offices, schools, shopping malls, hotels, public transport, cafes and restaurants, and ensures the door handles are hygienic.

### TECHNICAL FIELD

This invention is related to a door handle with disinfectant function that enables to clean the door handles used in public and private places such as hospitals, health care centers, public offices, schools, shopping malls, hotels, public transport, cafe's and restaurants and to ensure said handles are hygienic.

The disinfectant liquid is prevented from leaking during and after the disinfection process by means of the cylinder shield provided in the mechanism belonging to the door handle with disinfectant function subject to the invention. Moreover if preferred, the door can be opened using the door handle via the mechanism without carrying out the disinfection process, during the opening of the door with said door handle. Moreover the mechanism allows young children to be able to open the door without involuntarily starting the disinfection process. The door handle with disinfectant function subject to the invention has a single direction function and thereby the liquid material therein is used economically.

### PRIOR ART

Nowadays door handles that have human contact and that are used in living spaces of communities such as hospitals, shopping centers and public and private places need to be disinfected. Door handles are the primary critical areas that need to be disinfected, on which micro organisms leading to diseases are located. The microbes that accumulate on door handles stick to the surface and as bacteria that lead to diseases that cultivate in such areas are not disinfected, human health is negatively affected. Developments are available in the prior art regarding this issue.

In the Turkish Utility Model document numbered TR 2011/00571 of the prior art, a sterile mechanism with a door knob/handle that provides hygiene by cleaning the handles of toilet doors that are used in public places such as shopping malls, hotels, public transport, cafe's and restaurants is described. Disinfection process is provided via the UV lamp that is located in the structure that is fixed on the door or door frame. The solution in the related document is provided by UV light. In our invention liquid disinfectant is being applied. The disinfection process is carried out with a different technical method in comparison to our invention.

In the Chinese patent document numbered CN 87209106 which is another prior art document, a door handle is mentioned which disinfects the door handle by spraying automatic liquid via a disinfectant mechanism. However as the spray tube is located on the door handle in the related document and as the spraying process is carried out from the outside onto the door handle, liquid also leaks onto the floor. Moreover if preferred by the users, the liquid that is sprayed can be sprayed on the hands of the user rather than the door handle when is being opened, and thereby the door may not be opened via the door handle while at the same time carrying out the out the disinfection process.

In the present applications, and the documents given as reference above, the one way function and the prevention of leaking of the liquid onto the floor following the disinfection process, and the optional usage of the disinfection process is not mentioned in the door handle mechanisms.

### DESCRIPTION OF THE INVENTION

The door handle with disinfectant function that has been developed to reach the aims of the invention, has been illustrated in the attached figures.

According to these figures;

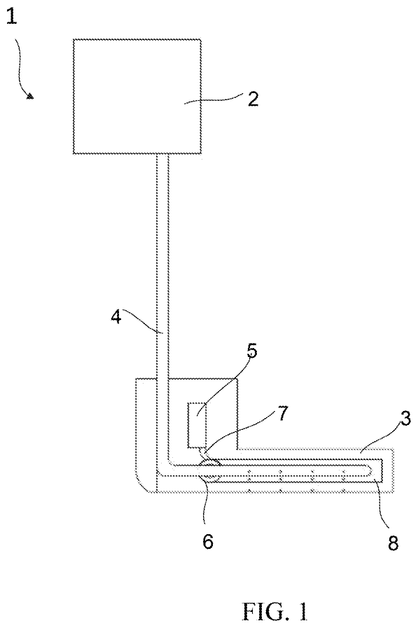

FIG. 1: The schematic view of the door handle with disinfectant function subject to the invention.

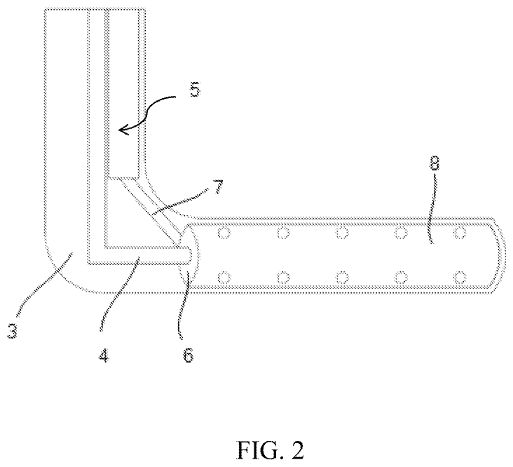

FIG. 2: The sectional view of the door handle with disinfectant function subject to the invention.

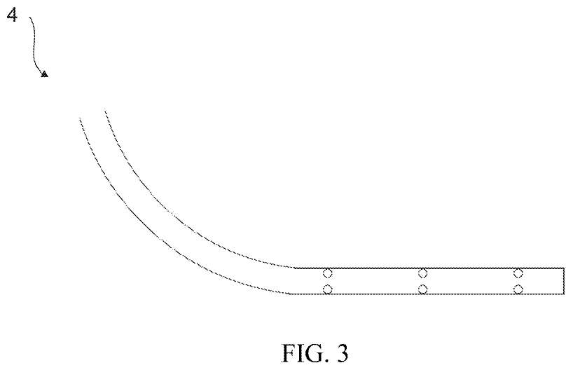

FIG. 3: View of the liquid transfer or delivery pipe of the door handle with disinfectant function subject to the invention.

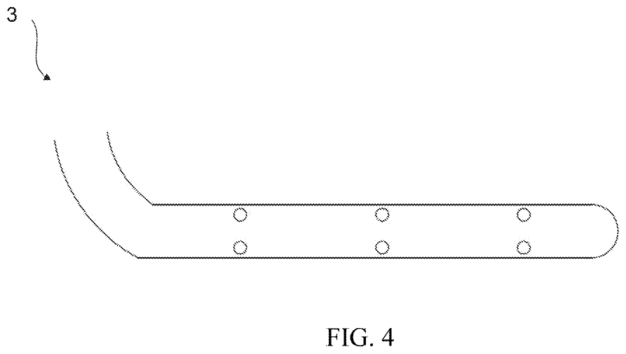

FIG. 4: External view of the door handle with disinfectant function subject to the invention.

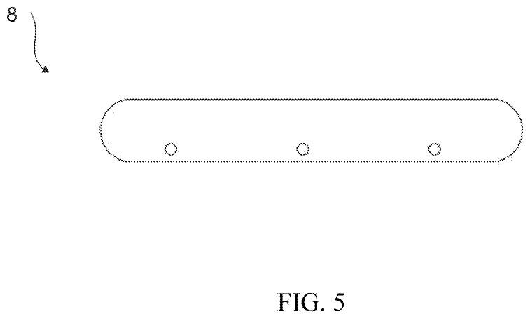

FIG. 5: View of the cylinder shield of the door handle with disinfectant function subject to the invention.

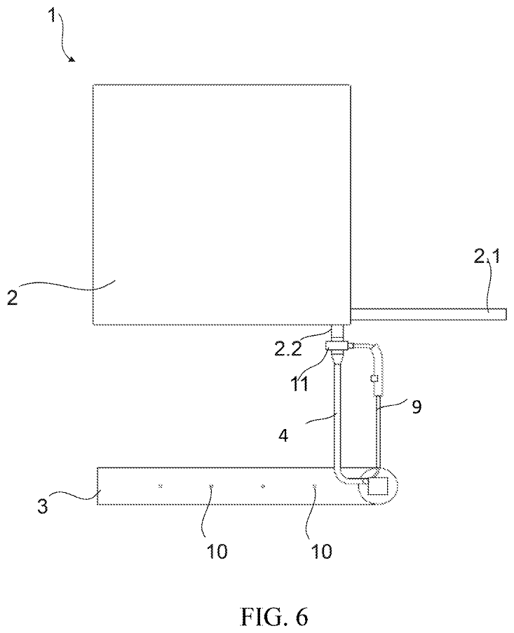

FIG. 6: The schematic view of the second application belonging to the operation principle between the door handle and the liquid tank of the door handle with disinfectant function subject to the invention.

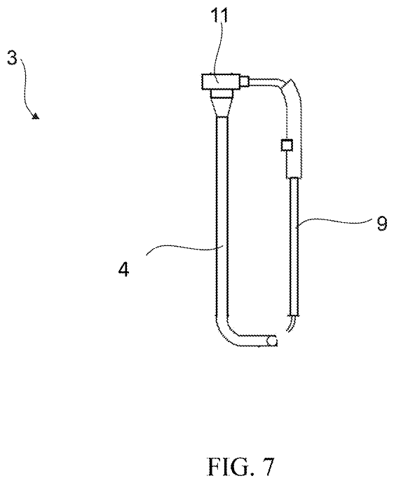

FIG. 7: View of the mechanism second application of the door handle with disinfectant function subject to the invention.

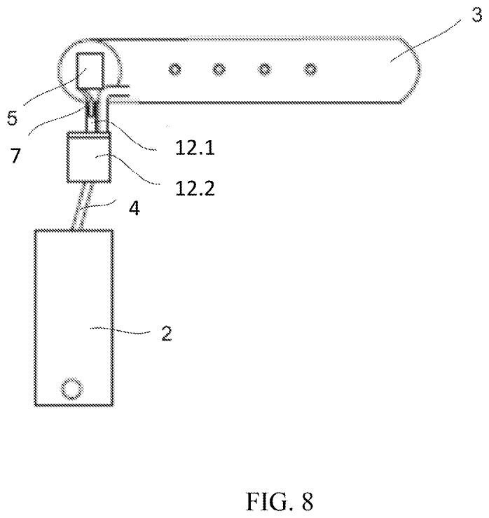

FIG. 8: Perspective view of the third application of the door handle with disinfectant function subject to the invention.

The parts that form the door handle with disinfectant function subject to the invention have been numbered as follows:
1. Door handle with disinfectant function
2. Liquid container
   2.1 Filling pipe
   2.2 Outlet opening
3. Handle
4. Delivery pipe
5. Door latch
6. Cylinder Cover
7. Lever (pressure lever)
8. Cylinder shield
9. Rope
10. Hole
11. Cover
12. Pump
   12.1 Cylinder
   12.2 Vacuum Tank

Door handle with disinfectant function (1) subject to the invention comprises;
A liquid container (2) that contains the disinfectant liquid that has a body which is embedded into the inner body of the door, a delivery pipe (4) having one end that is connected to the liquid container (2) and the other end leading to the inner section of said handle (3), that delivers the disinfectant liquid contained in the liquid container (2) to the door handle (3) that is mounted to the door, and a hole on the handle (3) that allows the disinfectant liquid contained in the container to be discharged from the external surface of the handle (3).

The door handle comprises a mechanism having a lever (7) whose one end is connected to the door latch (5) and the other to the cover (6), whereby when the handle (3) is turned down by the user, simultaneously the door latch (5) is activated and the cover (6) is rotated around its axis, to drive the mechanism to the open position and when the handle (3) is turned up by the user, the cover (6) is rotated in the reverse direction around its axis to drive the mechanism to the closed position; a cover (6) that covers the inlet opening of the cylinder shield (8), and it rotates the cylinder shield (8) at a calculated distance around its own axis via the force it receives from the lever (7) thereby aligning the holes of the cylinder shield and the holes of the door handle (3); and a cylinder shield (8) that is located at the inner section of the handle (3), that contains a delivery pipe (4) therein, with holes that are calculated and drilled at certain distances that on the external surface thereof, which rotates simultaneously with the rotation of the cylinder cover (6) and the holes thereof aligns with the holes on the door handle and as a result enables the disinfectant liquid contained in the delivery pipe (4) to flow on to the external surface of the door handle (3) and which also prevents the disinfectant liquid contained within the mechanism to leak out onto the external surface involuntarily.

The operation mechanism of the door handle with disinfectant function (1) subject to the invention comprises;
When the user turns the handle (3) in order to open the door, the lever (7) is activated and it delivers the force it receives from the door latch (5) to the cylinder cover (6).
The cylinder cover (6) receives the force from the lever (7) and it rotates simultaneously with the cylinder shield (8) by rotating around its own axis.
Following the simultaneous rotation of the cylinder shield (8), the holes of the cylinder shield (8) and the handle (3) are aligned and as a result, the disinfectant liquid contained inside the delivery pipe (4) that is in turn located in the cylinder shield (8) is enabled to be discharged from the surface of the handle (3).

As a result the handle (3) and the hand of the user are disinfected.

When the user does not wish to perform the disinfection process of the door handle with disinfectant function (1) subject to the invention, the user opens the door by turning the handle (3) up and the lever (7) is moved in the reverse direction. As a result, the cover (6) rotates in the reverse direction to the axis via the force it receives from the lever (7) and it also moves the cylinder shield (8) simultaneously in the reverse direction and therefore, the holes of the cylinder shield (8) and the handle (3) are no longer aligned and liquid discharge is onto the surface of the handle (3) is prevented and therefore the hand of the user and the handle (3) is not disinfected.

Another technical feature of the door handle with disinfectant function (1) subject to the invention is that when young children apply force on the handle (3), as the lever (7) within the handle (3) is not activated; the disinfectant mechanism is not operated. As a result young children can open the door without performing the disinfection process.

In the door handle with disinfectant function (1) subject to the invention the inner and outer handles move independent from each other. As a result when the handle (3) is opened from the inside, the mechanism at the outside is not operated and disinfectant liquid is not discharged from the handle (3) at the outside. Two separate delivery pipe (4) outlets are provided from the liquid container (2) of the door handle with disinfectant function (1) subject to the invention. As a result, different delivery pipes (4) feed the inner and outer door handles (3) of the mechanism.

Preferably the geometric structure of the cylinder shield (8) of the door handle with disinfectant function (1) subject to the invention is cylindrical. The cylinder shield (8) is located at the inner section of the handle (3). The cylinder shield (8) also has a delivery pipe (4) at its inner section. The disinfectant liquid that is discharged suitably from the liquid container is present in the delivery pipe (4) at all times. When the cylinder shield (8) rotates around its axis as a result of the force it receives from the cylinder cover (6), the holes on the shield and the door handle (3) are aligned and the liquid inside the delivery pipe (4) is released from the holes of the delivery pipe (4) and the liquid passes from the holes of the cylinder shield (8) and the holes of the handle (3) and is delivered to the outer surface of the handle (3). As a result the outer surface of the handle (3) is disinfected.

In another application of the invention the door handle comprises a liquid container that contains the disinfectant liquid that is mounted such that such that it is embedded into the inner section of the door body, having a filling pipe (2.1) that protrudes out of the front section of the door, allowing said disinfectant liquid to be fed to said filling pipe (2.1) from the outside and an outlet opening (2.2) at the bottom section, that allows discharging of the disinfectant liquid contained in said container (2), a cover (11) that is attached to the outlet opening (2.2), at least a rope (9), whose one end is attached to the cover (11) and the other end to the handle (3) whereby when the handle (3) is turned by the users, said rope tenses and it releases the cover (11), a mechanism having at least a delivery pipe (4) whose one end is connected to the outlet opening (2.2) and the other leading to the inner section of the handle (3), which delivers the disinfectant suitably from the liquid container (2) after the cover (11) is released, to the handle (3) and a handle (3) that is mounted to the related door, having at least a hole (10) that enables the disinfectant liquid that has been delivered from the delivery pipe (4) that is located at the inner section of the door, to be discharged onto the outer surface of the handle (3).

The liquid container (2) belonging to the door handle with disinfectant mechanism (1) subject to the invention has a filling pipe (2.1). When the disinfectant liquid within the liquid container (2) is low,

the process of loading disinfectant liquid into the filling pipe (2.1) is completed without having to assemble or disassemble the system by means of the liquid pipe (2.1).

The mechanism starts to operate simultaneously with the turning of the handle (3) of the door handle with disinfectant mechanism (1) subject to the invention.

The rope (9) is tensed and the cover (11) is released. As a result the disinfectant liquid is freely discharged from the liquid container (2) and the disinfectant liquid is delivered to the inner section of the handle (3) via the delivery pipe (4). The disinfectant liquid passes to the outside of the handle (3) via the holes (10) located on the handle (3) and thereby the outer surface of the handle (3) is disinfected.

Door handle system with disinfectant function (1) subject to the invention is mounted to both sides of the door. Preferably the all the other parts except the liquid container (2) are two of each. Ergonomic, efficient usage and aesthetic appearance is provided to the users by means of the door handle with disinfectant mechanism (1) subject to the invention.

In another application of the invention the door handle comprises, a liquid container (2) that is embedded to the inner section of the door body which contains the disinfectant liquid, a handle (3) having at least a hole that enables the disinfectant liquid to be discharged onto the outer surface of said handle, at least two delivery pipes (10) that extend to the inner section of the door handle (3) which enable the disinfectant liquid to be delivered to the inner section of the door handle, a pressure lever (7) that is connected from one end to the door latch (5) and the other end to the cylinder (12.1) which enables the mechanism to operate by being activated by the door latch (5) following the drive provided to the door handle (3) by the user, and a cylinder (12.1) whose one end is connected to the pressure lever (7) and the other end to the liquid container (2) via the delivery pipe (10), wherein the cylinder (12.1) moves vertically inside the vacuum tank (12.2), following the movement of the pressure lever (7) after the user turns the handle (3) and simultaneously drives the door latch (5), thereby compressing the liquid inside the vacuum tank which in turn creates a pressure effect and enables the liquid to be delivered to the handle (3) via the delivery pipe, and at least two pumps (12) provided with the a vacuum tank (12.2). In this application, when the users turn the door handle to open the door, the pressure lever (7) moves by means of the drive it receives from the door latch and the system of the door handle with disinfectant mechanism (1) is operated. The passage of the disinfectant liquid is enabled from the liquid container (2) via the delivery pipes to the pump (12) and from the pump to the inner section of the door handle (3) by means of the pressure created at the door handle with disinfectant mechanism (1) subject to the invention.

What is claimed is:

1. A door handle with a disinfectant function (1), characterized by, comprising;
a liquid container containing a disinfectant liquid, wherein the liquid container has a body embedded into an inner body of a door, a delivery pipe has a first end connected to the liquid container and a second end leading to an inner section of the door handle, wherein the delivery pipe delivers the disinfectant liquid contained in the liquid container to the door handle mounted to the door, and a hole on the door handle allows the disinfectant liquid contained in the liquid container to be discharged from an external surface of the door handle,
the door handle comprising a mechanism having a lever, wherein a first end of the lever is connected to a door latch and a second end of the lever is connected to a cylinder cover, whereby when the door handle is turned down by a user, simultaneously the door latch is activated and the cylinder cover is rotated around an axis of the cylinder cover, to drive the mechanism to an open position and when the door handle is turned up by the user, the cylinder cover is rotated in a reverse direction around the axis of the cylinder cover to drive the mechanism to a closed position;
the cylinder cover, wherein the cylinder cover covers an inlet opening of a cylinder shield, and the cylinder cover rotates the cylinder shield at a calculated distance around its own the axis of the cylinder cover via a force to align holes of the cylinder shield and holes of the door handle, and the cylinder cover receives the force from the lever;
and the cylinder shield, wherein the cylinder shield is located at the inner section of the door handle, and contains the delivery pipe in the cylinder shield, with the holes calculated and drilled at certain distances on an external surface of the cylinder shield, wherein the cylinder shield rotates simultaneously with a rotation of the cylinder cover and the holes of cylinder shield aligns with the holes on the door handle and as a result enables the disinfectant liquid contained in the delivery pipe to flow on to the external surface of the door handle and prevents the disinfectant liquid contained within the mechanism to leak out onto the external surface of the door handle involuntarily.

2. A door handle with a disinfectant function, comprising a liquid container, wherein the liquid container contains a disinfectant liquid and the liquid container is embedded into an inner section of a door body, a filling pipe is mounted to embed the filling pipe into the inner section of the door body and the filling pipe protrudes out of a front section of a door, and the filling pipe contains the disinfectant liquid allowing the disinfectant liquid to be fed to the liquid container from an outside and an outlet opening at a bottom section to allow discharging of the disinfectant liquid contained in the liquid container.

3. The door handle according to claim 2, comprising
a cover attached to the outlet opening, at least a rope, wherein a first end of the rope is attached to the cover and a second end of the rope is attached to the door handle, wherein when the door handle is turned by users, the rope tenses and the rope releases the cover,
a mechanism having at least a delivery pipe, wherein a first end of the delivery pipe is connected to the outlet opening and a second end of the delivery pipe is leading to an inner section of the door handle, the delivery pipe delivers the disinfectant liquid suitably from the liquid container after the cover is released, to the door handle, and
the door handle mounted to the door, wherein the door handle has at least a hole enabling the disinfectant liquid delivered from the delivery pipe located at an inner section of the door, to be discharged onto an outer surface of the door handle.

4. A door handle with a disinfectant function, comprising
a liquid container, wherein the liquid container is embedded to an inner section of a door body and contains a disinfectant liquid,
wherein the door handle comprises
at least a hole enabling the disinfectant liquid to be discharged onto an outer surface of the door handle,
at least two delivery pipes extend to an inner section of the door handle, wherein the at least two delivery pipes enable the disinfectant liquid to be delivered to the inner section of the door handle,
a pressure lever connected from a first end of the pressure lever to a door latch and a second end of the pressure lever to a cylinder for enabling a mechanism to operate by being activated by the door latch following a drive provided to the door handle by a user, wherein a first end of the cylinder is connected to the pressure lever and a second end of the cylinder is connected to the liquid container via the delivery pipe, wherein the cylinder moves vertically inside a vacuum tank, following a movement of the pressure lever after the user turns the door handle and simultaneously drives the door latch to compress the disinfectant liquid inside the vacuum tank for creating a pressure effect and enabling the disinfectant liquid to be delivered to the door handle via the delivery pipe, and
at least two pumps provided with the vacuum tank.
